(19) United States
(12) Patent Application Publication
(10) Pub. No.: US 2005/0058695 A1
(43) Pub. Date: Mar. 17, 2005

(54) Norethindrone sustained release formulations and methods associated therewith

(75) Inventors: Angela Anigbogu, Salt Lake City, UT (US); Ana Ruiz, Salt Lake City, UT (US); Danyi Quan, Salt Lake City, UT (US)

Correspondence Address: THORPE NORTH & WESTERN, P.O. BOX 1219, SANDY, UT, 84091-1219, US

(73) Assignee: Watson Pharmaccuticals, Inc.

(21) Appl. No.: 10/740,373

(22) Filed: Dec. 17, 2003

Related U.S. Application Data
(63) Continuation-in-part of application No. 10/449,861, filed on May 30, 2003.
Application No. 10/449,861 is a continuation-in-part of application No. PCT/US03/16933, filed on May 30, 2003.
(60) Provisional application No. 60/383,790, filed on May 30, 2002.

(51) Int. Cl. A61K 9/70; A61K 31/56

(52) U.S. Cl. 424/449; 514/170

(57) ABSTRACT

Sustained delivery formulations of norethindrone are disclosed and described. In one aspect, the formulation may be a transdermal formulation that includes both norethindrone and norethindrone acetate. In another aspect, the formulation may further include a penetration enhancer. Coadministration of norethindrone and norethindrone acetate has been found to provide a number of advantages, such as achievement of peak norethindrone serum levels substantially within 24 hours after initiation of administration.

### PRIORITY DATA

This application is a continuation-in-part of Patent Cooperation Treaty Application Serial No. PCT/US03/16933, filed on May 30, 2003, which claims priority to U.S. Provisional Patent Application Ser. No. 60/383,790, filed on May 30, 2002, each of which is incorporated herein by reference.

### FIELD OF THE INVENTION

The present invention relates to sustained release transdermal formulations of norethindrone and methods associated therewith. Accordingly, this invention covers the fields of pharmaceutical sciences, medicine and other health sciences.

### BACKGROUND OF THE INVENTION

Female hormones, such as estrogens and progestins have been indicated for a number of medicinal uses, such as hormone replacement therapy (HRT), and contraceptives for women. Both oral and transdermal dosage forms containing estrogens or progestins are well known, and often both are administered together in a single formulation. Due to the strict nature and perpetual duration of HRT and contraception, transdermal formulations are an attractive alternative to instant release oral dosage forms. However, because the skin is a formidable barrier for most drugs, transdermal administration typically requires a greater amount of time to attain significant onset of action, and provide the desired therapeutic effect.

One specific progestin that has received much attention is norethindrone (NE) and its prodrug norethindrone acetate (NEA). Both compounds have been transdermally administered as part of a number of specific formulations, including transdermal formulations. Examples of such formulations are contained in U.S. Pat. Nos. 5,211,952, 5,252,334, 5,422,119, 5,770,219, 5,783,208, 5,980,932, 6,149,935, 6,465,004, and U.S. Pat. Application Publication No. 2003/0152615, each of which is incorporated herein by reference.

As discussed in Applicant's copending patent application, U.S. Ser. No. 10/449,861, filed on May 30, 2003, which is incorporated herein by reference, a drawback to most transdermal formulations is that they fail to attain a maximum drug serum concentration in a rapid manner. For example, as illustrated by FIG. 3, of U.S. Pat. No. 5,980,932, maximum skin flux for NEA is attained only after the second day of administration. FIGS. 1 and 2 of U.S. Patent Application Publication No. 2003/0152615 shows a similar phenomenon where maximum NEA skin flux, is not obtained until nearly 48 following administration. As a result, the daily dosage received during the first day of administration may be inadequate to attain a needed effect. For example due to the strict dosing requirements posed by a contraception regimen, failure to attain required norethindrone blood levels during the first day of administration from a transdermal patch may be tantamount to skipping a day of administration in an oral dosage regimen and may increase the risk of pregnancy.

Another drawback that is experienced by many transdermal formulations, particularly adhesive matrix patches, is the inability to hold a sufficient amount of drug to provide therapeutic results over an extended duration, such as 7 days. In many cases, even if the matrix patch is able to hold enough drug to provide therapeutic results over an extended duration, the large amount of drug in the adhesive matrix may reduce the adhesiveness to a point where the patch will not adhere sufficiently to the skin for the intended duration of administration. Moreover, such high concentrations of drug in an adhesive matrix often cause the formation of drug crystal precipitating out of the adhesive.

Therefore, transdermal formulations of norethindrone that provide more rapid attainment of norethindrone serum levels, and which present improvements to other administration characteristics, such as efficacy, duration of administration, and stability, continue to be sought through ongoing research and development efforts.

### SUMMARY OF THE INVENTION

Accordingly, the present invention provides transdermal compositions and methods for the administration of female hormones to a subject. In one aspect, such a transdermal composition may include a pharmaceutically acceptable transdermal carrier, and a therapeutically effective amount of norethindrone and norethindrone acetate in the carrier.

The composition containing the norethindrone and norethindrone acetate may be formulated so as to provide a number of advantageous properties or results in accordance with the present invention. In one aspect, the composition may deliver the norethindrone and norethindrone acetate to a subject at a flux having a minimum peak rate of at least about 2.8 ug/cm²/hr. In another aspect, the composition may provide a norethindrone serum concentration that is sufficient to cause contraception in a female subject to which the composition is administered.

A number of pharmaceutically acceptable carriers are known to those of ordinary skill in the art and may be used in connection with the present invention. However, in one aspect, the carrier may be a polymeric adhesive matrix, such as a pressure sensitive adhesive. Typical transdermal matrix patches are known to employ a backing layer, a release liner, and a pressure sensitive adhesive layer adhered therebetween. In some aspects of the invention, the pressure sensitive adhesive layer may be made to include the therapeutically effective amount of norethindrone and norethindrone acetate, and to also have an adhesiveness that that is sufficient to remain in a drug transferring relationship on a skin surface of a subject for an administration period of from about 5 to about 7 days. In yet another aspect, the adhesive matrix layer may include an effective amount of norethindrone and norethindrone acetate and form substantially no crystals when stored at a temperature of from about 25° C. to about 40° C. and a relative humidity of from about 60% to about 75% for a period of from about 3 months to about 1 year.

The present invention additionally encompasses various methods of making and use associated with the transdermal compositions disclosed herein. In one aspect, the present invention includes a method of providing norethindrone therapy to a female subject which includes applying a transdermal composition as recited herein to a skin surface of the female subject. In another aspect, the norethindrone therapy may be contraception.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “an adhesive” includes reference to one or more of such adhesives, and reference to “an excipient” includes reference to one or more of such excipients.

As used herein, “estrogen,” “estrogenic steroid,” and “estrogenic hormone” may be used interchangeably, and refer to any substance, natural or synthetic, that exerts a biological or pharmacological action primarily by binding to estrogen receptors. Examples include but are not limited to: 17-β-estradiol, 17-β-estradiol, estriol, estrone, and phytoestrogens. These estrogens may be derivatized or modified to form various compounds. Examples of estrogens that may be used in the present invention include without limitation, conjugated equine estrogens, esterified estrogens, ethinyl estradiol, etc. Additional examples include but are not limited to: estradiol-3,17-diacetate, estradiol-3-acetate, estradiol-17-acetate, estradiol-3,17-divalerate, estradiol-3-valerate, estradiol-17-valerate. The estrogens may also be present as salts, (e.g., as sodium estrogen sulfate), isomers, or prodrugs.

As used herein in, “norethindrone,” or “NE” refers to a compound having the general chemical structure: 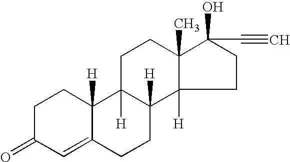
Norethindrone is well known in the art, and is listed as monograph 6790 on pg. 1149 of the Merck Index (12^{th} ed. 1996), which is incorporated herein by reference.

As used herein, “norethindrone acetate,” or “NEA” refers to a compound having the general chemical structure: 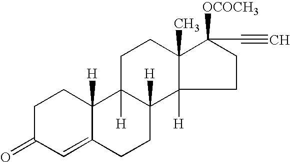
Norethindrone acetate is well known in the art as an ester-type prodrug of norethindrone and is described on pg. 1096 of Remington: The Science and Practice of Pharmacy (19^{th} ed. 1995), which is incorporated herein by reference.

As used herein, “subject” refers to a mammal that may benefit from the administration of a drug composition or method of this invention. Examples of subjects include humans, especially females, and may also include other animals such as horses, pigs, cattle, dogs, cats, rabbits, and aquatic mammals.

As used herein, the terms “formulation” and “composition” are used interchangeably. The terms “drug,” “pharmaceutical,” “active agent,” and “bioactive agent” are also used interchangeably to refer to a pharmacologically active substance or composition. These terms of art are well-known in the pharmaceutical and medicinal areas.

As used herein, the terms “administration,” and “administering” refer to the manner in which a drug is presented to a subject. Administration can be accomplished by various routes well-known in the art such as oral, and non-oral methods.

As used herein, “transdermal” refers to the route of administration that facilitates transfer of a drug through a skin surface wherein a transdermal composition is administered to the skin surface.

As used herein, “skin,” “skin surface,” “derma,” and “epidermis” may be used interchangeably, and are meant to include not only the outer skin of a subject comprising one or more of epidermal layers, but also to include mucosal surfaces to which a drug composition may be administered. Examples of mucosal surfaces include the mucosa of the respiratory (including nasal and pulmonary), oral (mouth and buccal), vaginal, and rectal cavities. Hence the terms “transdermal” may encompass “transmucosal” as well.

As used herein, “enhancement,” “penetration enhancement,” or “permeation enhancement,” refer to an increase in the permeability of the skin, to a drug, so as to increase the rate at which the drug permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of permeation enhancers is disclosed by David W. Osborne and Jill J. Henke, in their publication entitled Skin Penetration Enhancers Cited in the Technical Literature, published in “Pharmaceutical Technology” (June 1998), which is incorporated by reference herein.

An “effective amount” of an enhancer refers to an amount sufficient to increase penetration of a drug through the skin, to a selected degree. Methods for assaying the characteristics of permeation enhancers are well-known in the art. See, for example, Merritt et al., Diffusion Apparatus for Skin Penetration, J. of Controlled Release 61 (1984), incorporated herein by reference in its entirety. By “effective amount” or “therapeutically effective amount,” or similar terms is meant a non-toxic but sufficient amount of a drug, to achieve therapeutic results in treating a condition for which the drug is known to be effective. The determination of an effective amount is well-within the ordinary skill in the art of pharmaceutical and medical sciences. See for example, Curtis L. Meinert & Susan Tonascia, Clinical Trials: Design, Conduct, and Analysis, Monographs in Epidemiology and Biostatistics, vol. 8 (1986).

As used herein, “pharmaceutically acceptable carrier,” and “carrier” may be used interchangeably, and refer to any inert and pharmaceutically acceptable material that has substantially no biological activity, and makes up a substantial part of the formulation. The carrier may be polymeric, such as an adhesive, or non-polymeric and is admixed with other components of the composition (e.g., drug, binders, fillers, penetration enhancers, anti-irritants, emollients, lubricants, etc., as needed) to comprise the formulation.

The term “admixed” means that the drug and/or enhancer can be dissolved, dispersed, or suspended in the carrier.

By the term “matrix”, “matrix system”, or “matrix patch” is meant a composition comprising an effective amount of a drug dissolved or dispersed in a polymeric phase, which may also contain other ingredients, such as a permeation enhancer diluents, skin irritation reducing agents, excipients, plasticizers, emollients, and other optional ingredients. This definition is meant to include embodiments wherein such polymeric phase is a pressure sensitive adhesive, is laminated to a pressure sensitive adhesive, or is used within an overlay adhesive.

A matrix system may also comprise an adhesive layer having an impermeable film backing attached onto the distal surface thereof and, before transdermal application, a release liner on the proximal surface of the adhesive. The film backing protects the polymeric phase of the matrix patch and prevents release of the drug and/or optional ingredients to the environment. The release liner functions similarly to the impermeable backing, but is removed from the matrix patch prior to application of the patch to the skin as defined above. Matrix patches with the above-described general characteristics are known in the art of transdermal drug delivery. See, for example, U.S. Pat. Nos. 5,985,317, 5,783,208, 5,626,866, 5,227,169, which are incorporated by reference.

As used herein, “liquid reservoir system,” its acronym “LRS,” or “liquid reservoir patch” refers to a transdermal delivery patch or system, in which a drug and other optional ingredients, such as a permeation enhancer, are admixed with a fluid carrier vehicle of desired viscosity, such as a gel or ointment, which is formulated for confinement in a reservoir having an impermeable backing and a skin contacting permeable membrane, or membrane adhesive laminate providing diffusional contact between the reservoir contents and the skin. For application, a peelable release liner is removed and the patch is attached to the skin surface. LRS patches are known in the art of transdermal drug delivery. Examples without limitation, of LRS transdermal patches are those described or referred to in U.S. Pat. Nos. 4,849,224, 4,983,395, which are incorporated by reference in their entirety.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a concentration range of 0.1 to 5 should be interpreted to include not only the explicitly recited limits of 0.1 and 5, but also to include individual values such as 0.2, 0.7, 1.0, 2.2, 3.6, 4.2, and sub-ranges such as 0.3-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10”.

The Invention

As described above, the present invention provides transdermal compositions and methods for the delivery of female hormones, such as estrogens and progestins. It has been determined that norethindrone acetate does not convert to norethindrone in situ in the transdermal formulation, but rather, is only converted to norethindrone after it has migrated out of the formulation, and into the serum of a subject receiving the medication. Accordingly, Applicants have discovered that coadministration of norethindrone and norethindrone acetate to the skin of a subject c an yield various advantages over transdermal administration of either norethindrone or norethindrone acetate alone. For example, in one aspect, transdermal co-delivery of norethindrone and norethindrone acetate may result in a synergistic effect that attains a higher skin flux and norethindrone serum concentration in the subject, than is attained by delivering an equivalent amount norethindrone alone or norethindrone acetate alone.

In one aspect, the female hormones to be delivered may be norethindrone and norethindrone acetate. In another aspect, the female hormones may further include an estrogenic hormone. A number of estrogenic hormones may be suitable for use in the transdermal compositions of the present invention, and specific hormones may be selected by one of ordinary skill in the art in order to attain a particularly desired result. Examples of estrogenic hormones that can be used in the present transdermal formulations include without limitation, 17-β-estradiol, 17-α-estradiol, estriol, estrone, and phytoestrogens. These estrogens may be derivatized or modified to form various compounds. Conjugated equine estrogens, esterified estrogens, ethinyl estradiol, etc. may all be used in the present invention. Additionally, esterified estrogens including but are not limited to: estradiol-3,17-diacetate, estradiol-3-acetate, estradiol-17-acetate, estradiol-3,17-divalerate, estradiol-3-valerate, estradiol-17-valerate may be used. In a detailed aspect of the invention, the estrogenic hormone may be an estradiol. In another aspect, the estradiol may be ethinyl estradiol.

The specific amount of norethindrone and norethindrone acetate to be included in the transdermal formulations of the present invention may be a matter of choice depending on a specifically desired result to be achieved. However, in one aspect, the norethindrone amount may be from about 0.01% w/w to about 25% w/w of the formulation, and the norethindrone acetate amount may be from about 0.01% w/w to about 20% w/w. In a further aspect, the norethindrone amount may be from about 0.3% w/w to about 5% w/w of the formulation, and the norethindrone acetate amount may be from about 3% w/w to about 25% w/w of the formulation. In another aspect, the norethindrone amount may be from about 0.5% w/w to about 3% w/w, and the norethindrone acetate amount may be from about 3% w/w to about 12% w/w of the formulation. In yet another aspect, the norethindrone amount may be from about 1% w/w to about 2% w/w, and the norethindrone acetate amount may be from about 4% w/w to about 8% w/w of the formulation. In a further aspect, the norethindrone amount may be from about 1.5% w/w to about 2.5% w/w of the formulation. In an additional aspect, the norethindrone amount may be about 1% w/w of the formulation and the norethindrone acetate amount may be about 7.5% w/w of the formulation. In yet another aspect, the norethindrone amount may be about 0.5% w/w of the formulation, and the norethindrone acetate amount may be about 7.5% of the formulation. In a further aspect, the norethindrone amount may be about 1% w/w of the formulation and the norethindrone acetate may be about 15% w/w of the formulation.

Alternatively, it is possible to quantify the amount of norethindrone with respect to the amount of norethindrone in a formulation as a ratio. In one aspect, the norethindrone and norethindrone acetate may be present in the formulation at a weight ratio of from about 1:1 to about 1:25. In another aspect, the ratio may be from about 1:2 to about 1:8. In yet another aspect, the ration may be about 1:7.5 or 1:8. In another aspect, the ratio may be greater than about 1:10. In an additional aspect, the ratio may be about 1:15.

Additionally, the specific amount of estrogenic hormone to be included in the transdermal compositions of the present invention may vary based on a number of criteria. The specific estrogen to be delivered, the other components included in the formulation, and the serum concentrations or profiles to be obtained, may all be taken into consideration. However, in one aspect, the amount of estrogenic hormone may be sufficient to aid the norethindrone and norethindrone acetate in providing a contraceptive effect. In another aspect, the amount of estrogen may be from about 0.1% w/w to about 7% w/w of the composition. In another aspect, the amount of estrogen may be from about 0.5% w/w about 3% w/w. In a further aspect, the amount of estrogen may be from about 1% w/w to about 2% w/w. In yet another aspect, the amount of estrogen may be about 1% w/w of the composition.

In addition to percentages, the amount of estrogen included in the composition of the present invention may be calculated as a functional equivalent to a known amount of a specific estrogen species, such as ethinyl estradiol. Accordingly, in one aspect, the amount of estrogen included in the composition is an amount that provides a therapeutic effect that is substantially equivalent to an effect produced by about 0.1 to about 3 mg of a transdermally administered ethinyl estradiol. In another aspect, the amount of estrogenic hormone may be sufficient to provide a therapeutic effect that is substantially equivalent to an effect produced by about 1 mg of a transdermally administered ethinyl estradiol. In a further aspect, the amount of estrogenic hormone may be sufficient to provide a therapeutic effect that is substantially equivalent to an effect produced by about 0.75 mg of transdermally administered ethinyl estradiol. In yet another aspect, the amount of estrogenic hormone may be sufficient to provide a therapeutic effect that is substantially equivalent to an effect produced by about 0.5 mg of transdermally administered ethinyl estradiol. Such dosage amounts will typically be administered over a period of several days to a week with a small portion thereof actually being administered to the subject on a daily basis. Those of ordinary skill in the art will recognize a number of routine mechanisms for determining an estrogenic hormone amount that is sufficient to provide a therapeutic effect equivalent to a specified ethinyl estradiol concentration, and that such determinations may be readily made without undue experimentation.

In addition to the desired amount and number of bioactive agents, the transdermal formulations of the present invention may also optionally include a permeation enhancer, or mixture of permeation enhancers. A number of penetration enhancers may provide effective enhancement of the norethindrone and norethindrone acetate contained in the compositions of the present invention. By way of example without limitation, suitable penetration enhancers may include: fatty acids, fatty acid esters, fatty alcohols, fatty ethers, lower alcohols, glycerol esters, polyhydric alcohols, dials, amides, amines, terpenes, polar solvents, and mixtures thereof. In some aspects, the enhancer may be a member selected from the group consisting essentially of: C₁₀ to C₁₅ alcohols, isopropyl myristate, methyl laurate, oleyl alcohol, glycerol monooleate, glycerol dioleate, glycerol trioleate, glycerol monostearate, glycerol monolaurate, propylene glycol monolaurate, sorbitan esters, triacetin, propylene glycol, glycerol, ethanol, propanol, DMSO, dipropylene glycol, lauryl alcohol, dimethylformamide; N,N-dimethylacetamide; 2-pyrrolidone; N-(2-hydroxyethyl) pyrrolidone, N-methylpyrrolidone and mixtures thereof. In yet another aspect, the enhancer may be a C₁₀ to C₁₅ alcohol. In another aspect, the enhancer may be a lauryl-type or polyol-type enhancer.

The amount of the enhancer required to be effective may depend to some degree on the particular enhancer used, and one of ordinary skill in the art should be able to adjust the amount of a particular enhancer in order to provide an optimized formulation through routine experimentation. However, as a general matter, the amount of enhancer included in the transdermal formulation may be from about 0.01% w/w to about 50% w/w of the formulation. In a more detailed aspect, the amount of enhancer may be from about 3% w/w to about 16% w/w of the formulation. In a further aspect, the amount of enhancer may be about 8% w/w of the formulation. In an additional aspect, the amount of enhancer may be about 5% w/w of the formulation.

In one embodiment of the present invention, the composition may deliver the norethindrone and norethindrone acetate to a subject in accordance with a desired norethindrone and norethindrone acetate flux profile. In some instances it may be desirable to attain a flux profile with a peak flux rate of from at least about 2.0 ug/cm²/hr to at least about 3.2 ug/cm²/hr. However, in one aspect, the peak flux rate may be at least about 2.0 ug/cm²/hr. In another aspect, the peak flux rate may be at least about 3.0 ug/cm²/hr. In yet another aspect, the peak flux rate may be at least about 3.2 ug/cm²/hr. In another aspect, the minimum peak flux rate may be from about 1.0 to about 3.2 ug/cm²/hr. In an additional aspect, the minimum peak flux rate may be from about 1.5 to about 2.5 ug/cm²/hr. In a further aspect, the minimum peak flux rate may be from about 2.8 to about 4 ug/cm²/hr.

The compositions of the present invention may achieve the above-recited minimum peak flux rates at various times during an administration period. In one aspect, the minimum peak flux rate may be achieved by the composition within about 60 hours after initiation of administration. In another aspect, the minimum peak flux rate may be achieved by the composition within about 48 hours after initiation of administration. In an additional aspect, the minimum peak flux rate may be achieved by the composition within about 24 hours after initiation of administration. In a further aspect, the minimum peak flux rate may be achieved by the composition within a time period of from about 10 hours to about 60 hours. In some aspects, the time period may be from about 12 hours to about 24 hours. In other aspects, the time period may be from about 12 hours to about 36 hours. In yet other aspects, the time period may be from about 24 to about 36 hours. In additional aspects, the time period may be from about 8 hours to about 24 hours. In further aspects, the time period may be from about 8 hours to about 16 hours. In an additional aspect, the time period may be from about 24 to about 48 hours. In another aspect, the time period may be from about 36 to about 60 hours. In yet another aspect, the time period may be from about 36 to about 48 hours.

In each of these cases, the flux rates can also be sustained as required in order to provide a specifically desired result. In one aspect, the peak flux rate is followed by a subsequent flux rate of no less than about 40% of the peak flux rate until at least about 4-7 days after initiation of administration. In some cases the duration may be 4 days, and in others it may be 7 days. In another aspect, the subsequent flux rate may be no less than about 50% of the peak flux rate. In yet a further aspect, the subsequent flux rate may be no less than about 60% of the peak flux rate.

In another embodiment of the present invention, the composition may be delivered to a female for the purpose of contraception. In such a case, the composition delivers a therapeutically effective amount of norethindrone and norethindrone acetate to provide a norethindrone serum concentration that is sufficient to cause contraception. Various minimum norethindrone serum levels may provide effective contraception in different female subjects. However, in one aspect, the minimum norethindrone serum concentration provided may be at least about 150 pg/ml. In another aspect, the minimum norethindrone serum concentration provided may be at least about 250 pg/ml. In a further aspect, the minimum norethindrone serum concentration provided may be from about 150-1500 pg/ml. In yet another aspect, the minimum norethindrone serum concentration provided may be at least about 500 pg/ml.

One measure of serum levels that are sufficient to provide effective contraception may be found in an evaluation of currently known oral norethindrone contraceptives. For example, one currently FDA approved progestin only oral contraceptive is marketed under the trade name NOR-QD by Watson Pharmaceuticals Inc., Corona, Calif. In order to provide effective contraception, this product contains 0.35 mg of norethindrone to be orally administered on a once daily basis. Additional product information may be found on pg. 3423 of The Physician's Desk Reference, 56^{th} ed. (2002), which is incorporated herein by reference. Accordingly in one aspect, the norethindrone serum level provided by the composition of the present invention may be substantially equivalent to a serum level provided by a 0.35 mg oral dose of norethindrone.

The transdermal composition of the present invention may be formulated to attain the necessary norethindrone serum concentrations at specific pre-designed time points, and to maintain such serum concentration for given durations. In one aspect, the composition may achieve the desired norethindrone serum concentration within about 48 hours after initiation of administration. In another aspect, the desired norethindrone serum concentration may be attained within about 24 hours after initiation of administration. In a further aspect, the minimum norethindrone serum concentrations may be sustained for a duration of from about 4 to about 7 days. In some aspects the duration may be at least about 4 days. In other aspects, the duration may be from about 5 to about 7 days. In yet additional aspects, the duration may be at least about 7 days.

The transdermal formulation of the present invention may take the form of an occlusive device, such as a transdermal patch. Such a transdermal patch may either be an adhesive matrix patch, a liquid reservoir system type patch, a buccal tablet, or the like. A wide variety of each specific device type are known to those of ordinary skill in the art. Optional ingredients such as adhesives, excipients, backing films, release liners, etc., and the required amount of each will vary greatly depending upon the type of patch desired, and may be determined as needed by one ordinarily skilled in the art to arrive at a specific formulation with desired characteristics and properties.

In one aspect of the present invention the formulation may be a transdermal adhesive matrix patch. In some aspects, such matrix patches may include a backing member, a polymeric adhesive matrix, and the active agents. A removable protective release liner may be provided to protect the drug-containing adhesive matrix until ready for use. Monolithic systems where the drug is contained directly in a single pressure sensitive adhesive layer, as well as systems containing one or more polymeric reservoirs in addition to the pressure sensitive adhesive layer may be used. As noted above, in one aspect of the present invention, a permeation enhancer may be used to increase the delivery rate of the drug, and may also be used to vary other parameters, such as patch size, etc.

Transdermal matrix patches are known in a variety of sizes. Size for a transdermal matrix patch is most often correlated to the area of the adhesive layer that contacts the skin and administers the drug contained in the patch. Patches ranging in size of from about 5 cm² to about 200 cm² have been known. In one aspect, the transdermal matrix patch of the present invention may have a drug delivering skin contact surface of from about 5 cm² to about 50 cm². In another aspect, the drug delivering skin contact surface may be from about 10 cm² to about 35 cm². In a further aspect, the drug delivering skin contact surface may be from about 15 cm² to about 25 cm². In an additional aspect, the drug delivering skin contact surface may be from about 5 cm² to about 13 cm². In a further aspect, the drug delivering skin contact surface may be from about 13 cm² to about 18 cm². In another aspect, the drug delivering skin contact surface may be from about 13 cm² to about 39 cm². In yet another aspect, the drug delivering skin contact surface may be from about 13 cm² to about 26 cm². In a further aspect, the drug delivering skin contact surface may be from about 18 cm² to about 36 cm².

Polymeric adhesives suitable for use in the present invention may include, but are not limited to, crosslinked or uncrosslinked acrylic copolymers (e.g. DUROTAK 87-2516, 87-2074, 87-2979, 87-9301 etc. National Starch and Chemical Co.), acrylics, vinyl acetates, natural and synthetic rubbers, ethylenevinylacetate copolymers, polysiloxanes, polyacrylates, polyurethanes, polyisobutylene copolymers, polyether block amide copolymers, and mixtures thereof. Those of ordinary skill in the art will appreciate that the specific type and amount of adhesive polymer used may be selected depending upon the desired specific characteristics of the final product. However, in one aspect, the adhesive matrix may be an acrylic adhesive that is substantially free of vinyl acetate and functional monomers. Such an adhesive is sold under the trade name DUROTAK 87-9301 by National Starch and Chemical Co. In another aspect, the polymeric adhesive may consist essentially of an acrylic adhesive.

In one aspect, the amount of adhesive polymer in the adhesive matrix layer may be at least about 50% w/w of the adhesive layer. In another aspect, the amount may be at least about 60% w/w of the adhesive layer. In yet another aspect, the amount may be at least about 85% w/w of the adhesive layer. In a further aspect, the amount may be at least about 90% w/w of the adhesive layer. In an additional aspect, the amount may be from about 50% w/w to about 95% w/w of the adhesive layer.

Those of ordinary skill in the art will appreciate the difficulty often encountered in attempting to ascertain an adhesive for use as a carrier that provides optimal drug solubility characteristics and optimal adhesive characteristics. Often adhesiveness is compromised in order to obtain a sufficient drug load for extended administration, or else drug load is compromised in order to retain sufficient adhesiveness for an extended administration. However, in accordance with one aspect of the present invention, a transdermal matrix patch may be provided with a pressure sensitive adhesive layer that includes a therapeutically effective amount of norethindrone and norethindrone acetate, and has an adhesiveness that is sufficient to remain in a drug transferring relationship with a skin surface of a subject for an administration of from about 5-7 days. In some aspects, the administration period may be at least about 7 days. A variety of the above-recited adhesives, including combinations and blends of particular species thereof, may provide both the requisite norethindrone and norethindrone acetate solubility characteristics, as well as the adhesiveness characteristics required to remain in a drug transferring relationship on the skin and provide effective norethindrone and norethindrone acetate administration for the extended durations recited herein.

As noted above, in accordance with the present invention, the drug-containing adhesive matrix layer can, in addition to the polymeric adhesive matrix and drug, also contain other optional ingredients, such as carriers, vehicles, permeation enhancers, excipients, diluents, emollients such as glycerin, and the like, which are suitable for administration in conjunction with the present invention. Such materials are pharmaceutically acceptable in that they are nontoxic, do not hinder drug delivery, and are not for any other reasons biologically or otherwise undesirable. Examples of such additional materials include water, mineral oil, silicone, inorganic gels, aqueous emulsions, liquid sugars, waxes, petroleum jelly, and a variety of other oils and polymeric materials. Those of ordinary skill in the art will be able to select specific additives in specific amounts in order to provide a matrix patch with particularly desired characteristics.

In another aspect of the present invention, polymers useful for the backing layer are polyethylene, polypropylene, polyesters, polyurethanes, polyethylene vinyl acetate, polyvinylidene chloride, block copolymers such as PEBAX, and the like.

The formulations of the present invention include sustained release formulations that administer therapeutically effective amounts of norethindrone and norethindrone over an extended period of time. However, in one aspect, a sustained delivery the sustained delivery period of norethindrone and norethindrone acetate may be for at least 7 days. In another aspect, the sustained delivery period may be at least 5 days. In a further aspect, the sustained delivery period may be at least 3 days.

In addition to the transdermal formulations and compositions disclosed herein, the present invention includes methods for the making and use thereof. In one aspect, a norethindrone serum concentration in a subject that was achieved by transdermal delivery of either norethindrone alone or norethindrone acetate alone may be exceeded by transdermally administering an equivalent combined amount of norethindrone and norethindrone acetate. In another aspect, a maximum norethindrone serum concentration may be achieved in a subject within about 24, or about 36, or from about 24 to about 36 hours after initiation of transdermal administration by delivering a combination of norethindrone and norethindrone acetate. In yet another aspect, norethindrone and norethindrone acetate permeation through the skin of the subject may be enhanced by utilizing the enhancers enumerated herein.

In addition to the foregoing properties and characteristics, the present invention also provides a transdermal matrix patch having a pressure sensitive adhesive layer that includes a therapeutically effective amount of norethindrone and norethindrone acetate and which forms substantially no crystals when stored at a temperature of from about 25° C. to about 40° C. and a relative humidity of from about 60% to about 75% for a period of from about 3 months to about 1 year. In one aspect, the temperature may be about 40° C. and the relative humidity may be about 75%, with a storage period of at least about 3 months. In another aspect, the temperature may be about 30° C. and the relative humidity may be about 70%, and the storage period may be at least about 6 months. In a further aspect, the temperature may be about 25° C. and the relative humidity may be about 60%, and the storage period may be at least about 9 months. Alternative periods of storage may be used for each temperature and humidity condition. Stability testing of this sort, and for other issues, such as degradation products, pathogen presence, etc. is well known in the pharmaceutical arts and mandated in order to receive FDA product approval.

### EXAMPLES

The following examples of norethindrone formulations are provided to promote a more clear understanding of certain embodiments of the present invention, and are in no way meant as a limitation thereon.

### Example 1

Transdermal matrix systems containing norethindrone and norethindrone acetate were made as follows. The solids contents of an acrylic adhesive solution, (Durotak 87-2074) was determined by placing small amounts into pre-weighed aluminum dishes which were then put in a convection oven (Model A4718-Q, Blue M) at 75° C. overnight. Following evaporation of the solvents, the weight of the dry adhesive was obtained and the solids content calculated as a ratio of the dry to wet weight.

The adhesive 87-2074 contains approximately 28-31% solids and was always used undiluted. Known quantities of the adhesive were weighed into glass bottles based on the previously determined solids content. For all the formulations, appropriate quantities of norethindrone (NE) were first added to the liquid adhesive in each bottle (to give 1% w/w drug content upon drying). The bottles were capped and sealed with parafilm and rotated until all the NE was dissolved. Appropriate quantities of norethindrone acetate (NEA) (to give 7.5% w/w drug upon drying) were added to the bottle of the formulation in which no enhancer is desired. To the other bottles, appropriate quantities of norethindrone acetate and lauryl alcohol or 1-lauryl-2-pyrrolidone (LP-300) or mixtures of lauryl alcohol and 1-lauryl-2-pyrrolidone (LP-300) were added to the bottles containing norethindrone in adhesive to give the desired compositions upon drying. Each bottle was again tightly capped, sealed with parafilm and rotated overnight during which time the NE or NEA and enhancers had dissolved to yield a clear solution.

A small amount of each formulation (about 10 g) was placed onto the high release side of a silicone release-coated 3 Mil thick polyester (PET) liner (Loparex Inc., 10393S) and manually cast with a 10 Mil gap casting knife. Each cast was placed in a convection oven (Model A4718-Q, Blue M) at 75° C. for 15 minutes. After drying, each cast was laminated with a 3 Mil polyethylene (PET) monolayer backing film (3M, CoTran™ 9720).

### Example 2

Utilizing adhesive matrix patches made in accordance with the above-recited procedure, in vitro skin flux studies were conducted using modified Franz diffusion cells. Heat separated human cadaver epidermal membranes were used. The matrix patches for each formulation were cut into 0.71 cm² circular discs. The release liner was peeled and discarded and the matrix disc laminated onto the stratum corneum side of the epidermal membrane. The skin-matrix assembly was then sandwiched between the donor and receiver chambers of a diffusion cell and clamped in place with the epidermal side facing the receiver compartment. The receiver compartment was filled with 0.02% w/v sodium azide (NaN₃) solution. The cells were then placed in a circulating water bath maintained at 32±1° C.

At time points of 24, 48, 72, 96, 120, 144 and 168 hrs, the entire contents of the receiver compartment were collected for drug quantitation. The receiver compartment was then re-filled with fresh receiver medium. The interval flux and cumulative, or combined amounts of drug permeating per unit area were calculated following HPLC analyses of the samples. The flux study results are contained in Tables 1 and 2 below.

TABLE 1
A comparison of transdermal matrix formulations with and without
lauryl-type enhancers
DAILY DELIVERY (μg/cm²)

| NO | | | |
|---|---|---|---|
| ENHANCER | 5% LA | 3% LP-300 | 5% LA, 3% LP-300 |
| (Mean ± sd)* | (Mean ± sd)** | (Mean ± sd)* | (Mean ± sd)* |
| 2074/NEA/NE | 2074/NEA/NE/LA | 2074/NEA/NE/LP-300 | 2074/NEA/NE/LA/LP-300 |
| 91.5/7.5/1 | 86.5/7.5/1/5 | 88.5/7.5/1/3 | 83.5/7.5/1/5/3 |
| 10.9 ± 3.1 | 23.9 ± 6.1 (219%) | 25.7 ± 9.6 (236%) | 29.7 ± 8.5 (272%) |
| 10.6 ± 3.0 | 20.3 ± 4.7 (192%) | 19.8 ± 6.2 (187%) | 22.7 ± 6.8 (214%) |
| 9.6 ± 2.4 | 17.2 ± 4.4 (179%) | 16.8 ± 5.2 (175%) | 18.4 ± 5.0 (192%) |
| 8.7 ± 2.0 | 16.4 ± 3.0 (189%) | 16.4 ± 4.7 (189%) | 18.0 ± 4.5 (207%) |
| 7.7 ± 2.1 | 16.2 ± 3.6 (210%) | 16.7 ± 5.0 (217%) | 17.6 ± 3.8 (229%) |
| 7.3 ± 1.6 | 15.1 ± 3.3 (207%) | 15.2 ± 4.6 (208%) | 15.9 ± 3.2 (218%) |
| 7.6 ± 1.4 | 14.2 ± 3.1 (187%) | 14.6 ± 3.7 (192%) | 14.7 ± 2.8 (193%) |
| Average | 198 | 205 | 218 |
| Enhancement | | | |

*n = 6
**n = 3

TABLE 2
A comparison of transdermal formulations with and without
polyol-type enhancers

| | Daily Delivery (ug/cm²) | |
|---|---|---|
| | NO ENHANCER (Mean ± sd)* | 8% DPG (Mean ± sd)* 2074/ |
| DAY | 2074/NEA/NE 91.5/7.5/1 | NEA/NE/DPG 83.5/7.5/1/8 |
| DAY 1 | 10.9 ± 3.1 | 22.4 ± 8.2 (206%) |
| DAY 2 | 10.6 ± 3.0 | 16.0 ± 6.1 (151%) |
| DAY 3 | 9.6 ± 2.4 | 13.1 ± 4.7 (136%) |
| DAY 4 | 8.7 ± 2.0 | 12.8 ± 3.7 (147%) |
| DAY 5 | 7.7 ± 2.1 | 12.9 ± 3.8 (168%) |
| DAY 6 | 7.3 ± 1.6 | 11.2 ± 2.9 (153%) |
| DAY 7 | 7.6 ± 1.4 | 11.0 ± 2.6 (145%) |
| | Average enhancement | 158 |

*n = 3

As can be seen from the above-recited results, each of the enhancers tested showed significant increases in penetration as compared to formulations containing no enhancer. Further, as can be seen by both the formulations with and without enhancers, the combination of NE and NEA in all cases produced peak flux rates within the first 24 hours following initiation of administration, with flux rate gradually declining over the sustained release period of 7 days. As such, it can be concluded that the combination of NE and NEA will provide maximum norethindrone serum concentrations within about 24 hours of administration initiation, and will further provide effective norethindrone plasma concentrations over a sustained release period of at least 7 days.

### Example 3

The protocol of Example 1 was utilized in order to create transdermal adhesive matrix patches, except that a either Durotak 2074, or 2979 adhesive was used, and formulations were made to contain either 6% NEA by itself, or approximately 4% w/w of NEA and either 2% or 2.5% w/w NE. Skin flux experiments were carried out in accordance with the protocol of Example 2. The cumulative skin permeation results are contained in Tables 3-6 below.

TABLE 3
Cumulative Permeation of Norethindrone and Norethindrone Acetate
with 2979 Adhesive and Lauryl Alcohol

| Formulation | Donor ID | 24 hr | 48 hr | 72 hr | 96 hr |
|---|---|---|---|---|---|
| 2979/NEA/LA | Skin 1 | 18.12 ± 1.64 | 34.04 ± 2.93 | 48.63 ± 4.34 | 62.75 ± 5.55 |
| 86.5/6/7.5 | Skin 2 | 18.77 ± 1.03 | 35.40 ± 1.31 | 50.72 ± 2.13 | 64.87 ± 2.63 |
| | Skin 3 | 14.28 ± 1.25 | 28.40 ± 2.47 | 41.38 ± 3.70 | 53.96 ± 4.89 |
| | Mean | 17.06 ± 2.39 | 32.62 ± 3.81 | 46.91 ± 5.25 | 60.53 ± 6.42 |
| | (n = 3 | | | | |
| | skins) | | | | |
| 2979/NEA/NE/LA | Skin 1 | 28.96 ± 0.66 | 52.35 ± 0.70 | 73.05 ± 1.75 | 92.08 ± 2.70 |
| 86.5/4/2/7.5 | Skin 2 | 27.35 ± 5.11 | 49.91 ± 9.90 | 69.18 ± 13.99 | 86.47 ± 17.02 |
| | Skin 3 | 23.49 ± 4.14 | 46.48 ± 7.38 | 66.73 ± 9.93 | 86.50 ± 12.51 |
| | Mean | 26.60 ± 4.20 | 49.58 ± 6.93 | 69.65 ± 9.41 | 88.35 ± 11.46 |
| | (n = 3 | | | | |
| | skins) | | | | |

TABLE 4
Cumulative Permeation of Norethindrone and Norethindrone Acetate
with 2979 Adhesive, Lauryl Alcohol and Dipropylene Glycol

| Formulation | Donor ID | 24 hr | 48 hr | 72 hr | 96 hr |
|---|---|---|---|---|---|
| 2979/NEA/LA/DPG | Skin 1 | 18.23 ± 2.21 | 33.84 ± 3.91 | 48.28 ± 5.10 | 61.81 ± 6.45 |
| 79/6/7.5/7.5 | Skin 2 | 17.88 ± 5.32 | 33.34 ± 10.32 | 47.95 ± 14.60 | 61.49 ± 18.29 |
| | Skin 3 | 13.86 ± 2.57 | 27.80 ± 4.42 | 40.64 ± 6.11 | 52.66 ± 7.34 |
| | Mean | 16.66 ± 3.89 | 31.66 ± 6.83 | 45.62 ± 9.43 | 58.65 ± 11.70 |
| | (n = 3 | | | | |
| | skins) | | | | |
| 2979/NEA/NE/DPG/LA | Skin 1 | 38.16 ± 4.89 | 67.95 ± 8.39 | 94.43 ± 11.42 | 118.27 ± 13.67 |
| 79/4/2/7.5/7.5 | Skin 2 | 44.41 ± 8.78 | 79.12 ± 13.86 | 107.83 ± 17.19 | 132.60 ± 21.33 |
| | Skin 3 | 29.77 ± 7.14 | 57.51 ± 11.17 | 82.63 ± 14.42 | 107.30 ± 17.19 |
| | Mean | 37.45 ± 8.98 | 68.20 ± 13.81 | 94.96 ± 16.99 | 119.39 ± 19.30 |
| | (n = 3 | | | | |
| | skins) | | | | |

TABLE 5
Cumulative Permeation of Norethindrone and Norethindrone Acetate
with 2979 Adhesive, Lauryl Alcohol & Lauryl Pyrrolidone

| Formulation | Donor ID | 24 hr | 48 hr | 72 hr | 96 hr |
|---|---|---|---|---|---|
| 2979/NEA/LA/LP-300 | Skin 1 | 11.70 ± 1.31 | 23.66 ± 2.45 | 35.28 ± 3.85 | 45.61 ± 5.01 |
| 83/7/7/3 | Skin 2 | 17.87 ± 0.36 | 33.88 ± 0.70 | 50.08 ± 1.45 | 65.34 ± 1.74 |
| | Skin 3 | 18.50 ± 3.33 | 37.80 ± 7.54 | 57.17 ± 11.71 | 75.35 ± 15.60 |
| | Mean | 15.86 ± 3.85 | 31.59 ± 7.82 | 47.28 ± 12.05 | 61.81 ± 16.22 |
| | (n = 3 | | | | |
| | skins) | | | | |
| 2979/NEA/NE/LA/LP-300 | Skin 1 | 17.91 ± 1.84 | 35.36 ± 4.01 | 51.60 ± 6.52 | 65.95 ± 8.54 |
| 83.5/4/2.5/7/3 | Skin 2 | 27.90 ± 7.74 | 52.22 ± 14.14 | 75.36 ± 20.04 | 95.77 ± 24.54 |
| | Skin 3 | 28.19 ± 2.82 | 54.31 ± 5.77 | 79.63 ± 8.11 | 102.08 ± 9.60 |
| | Mean | 24.37 ± 6.45 | 46.85 ± 11.77 | 68.27 ± 17.05 | 87.22 ± 21.47 |
| | (n = 3 | | | | |
| | skins) | | | | |

TABLE 6
Cumulative Permeation of Norethindrone and Norethindrone Acetate
with 2074 Adhesive, Lauryl Alcohol & Lauryl Pyrrolidone

| Formulation | Donor ID | 24 hr | 48 hr | 72 hr | 96 hr |
|---|---|---|---|---|---|
| 2074/NEA/LA/LP-300 | Skin 1 | 8.40 ± 0.90 | 16.88 ± 1.51 | 25.03 ± 2.00 | 32.29 ± 2.43 |
| 83/7/7/3 | Skin 2 | 16.40 ± 2.20 | 31.95 ± 4.47 | 47.31 ± 6.91 | 62.27 ± 9.32 |
| | Skin 3 | 14.24 ± 1.69 | 28.41 ± 2.73 | 42.49 ± 4.03 | 55.97 ± 5.16 |
| | Mean | 13.01 ± 3.84 | 25.74 ± 7.30 | 38.28 ± 10.89 | 50.18 ± 14.64 |
| | (n = 3 | | | | |
| | skins) | | | | |
| 2074/NEA/NE/LA/LP-300 | Skin 1 | 13.69 ± 1.73 | 27.03 ± 3.30 | 39.67 ± 4.62 | 50.60 ± 5.59 |
| 83.5/4/2.5/7/3 | Skin 2 | 27.46 ± 6.20 | 50.30 ± 10.23 | 71.52 ± 13.92 | 90.28 ± 16.25 |
| | Skin 3 | 33.42 ± 2.76 | 60.17 ± 4.16 | 83.63 ± 6.11 | 102.95 ± 8.05 |
| | Mean | 24.08 ± 9.37 | 44.53 ± 15.73 | 63.24 ± 21.13 | 79.31 ± 25.44 |
| | (n = 3 | | | | |
| | skins) | | | | |

As can be seen, from Tables 3-6, in each case the formulations containing a combination of NE and NEA achieved superior flux results as compared to formulations containing the substantially the same total amount of NEA alone.

### Example 4

Additional transdermal patches were made using the general protocol of Example 1, except that a Durotak 87-9301 adhesive was used, and either 9% NEA alone, or 7.5% NEA and 1.5% NE, or 10% and 2% respectively were used. The formulations were tested according to the skin flux testing protocol enumerated in Example 2. The daily skin flux results are shown in Tables 7 and 8 below.

TABLE 7
Daily Delivery of Norethindrone and Norethindrone acetate with
Lauryl Alcohol as a Penetration Enhancer for 7 Day Administration

| | DAILY DELIVERY (μg/cm²⁾ | | | | | | |
|---|---|---|---|---|---|---|---|
| | TIME = 1 | TIME = 2 | TIME = 3 | TIME = 4 | TIME = 5 | TIME = 6 | TIME = 7 |
| | DAY | DAYS | DAYS | DAYS | DAYS | DAYS | DAYS |
| 9301/NEA/LA | | | | | | | |
| 86/9/5 | | | | | | | |
| GRAND MEAN | 10.85 | 10.37 | 9.06 | 7.78 | 7.02 | 7.66 | 6.99 |
| STD DEV | 5.52 | 5.28 | 4.22 | 4.00 | 3.19 | 3.81 | 3.28 |
| 9301/NEA/NE/LA | | | | | | | |
| 86/7.5/1.5/5 | | | | | | | |
| GRAND MEAN | 23.91 | 22.33 | 19.02 | 15.40 | 14.74 | 14.09 | 12.95 |
| STD DEV | 12.50 | 10.19 | 7.73 | 6.71 | 6.32 | 5.58 | 4.72 |
| Enhancement | 220% | 215% | 210% | 198% | 210% | 184% | 185% |

TABLE 8
Daily Delivery of Norethindrone and Norethindrone acetate with
Dipropylene Glycol as a Penetration Enhancer for 7 Day Administration

| | DAILY DELIVERY (μg/cm²) | | | | | | |
|---|---|---|---|---|---|---|---|
| | TIME = 1 | TIME = 2 | TIME = 3 | TIME = 4 | TIME = 5 | TIME = 6 | TIME = 7 |
| | DAY | DAYS | DAYS | DAYS | DAYS | DAYS | DAYS |
| 9301/NEA/DPG | | | | | | | |
| 86/9/5 | | | | | | | |
| GRAND MEAN | 7.35 | 6.00 | 5.73 | 5.12 | 4.93 | 4.90 | 5.69 |
| STD DEV | 1.90 | 1.77 | 1.78 | 2.18 | 1.71 | 2.09 | 1.64 |
| 9301/NEA/NE/DPG | | | | | | | |
| 86/7.5/1.5/5 | | | | | | | |
| GRAND MEAN | 15.33 | 11.37 | 10.36 | 8.71 | 8.80 | 8.23 | 9.11 |
| STD DEV | 6.65 | 5.07 | 4.22 | 2.95 | 3.42 | 3.35 | 2.78 |
| Enhancement | 209% | 190% | 181% | 170% | 178% | 168% | 160% |
| 9301/NEA/NE/DPG | | | | | | | |
| 83/10/2/5 | | | | | | | |
| GRAND MEAN | 17.96 | 13.61 | 12.28 | 10.25 | 10.50 | 10.12 | 10.68 |
| STD DEV | 6.16 | 5.08 | 4.55 | 3.55 | 3.93 | 3.76 | 3.52 |
| Enhancement | 244% | 227% | 214% | 200% | 213% | 207% | 188% |

As with the results obtained in Examples 2 and 3 above, the flux data for this example shows that when NE and NEA are combined into a single formulation, that a significant improvement in flux is achieved as opposed to substantially the same, or a similar total amount of NEA alone.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements.

Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A transdermal matrix patch for administration to a female subject comprising:
a backing layer;
a removable release liner; and
a pressure sensitive adhesive layer with a drug delivering skin contact surface area of from about 10 cm² to about 35 cm² adhered between the backing layer and the release liner, said pressure sensitive adhesive layer having an adhesiveness that, when transdermally applied to a skin surface of said subject, is sufficient to remain in a drug transferring relationship on the skin for an administration period of about 7 days,
said pressure sensitive adhesive layer having dispersed therein optionally a permeation enhancer, and norethindrone in an amount of from 0.3% to about 5% w/w of the adhesive layer, and norethindrone acetate in an amount of from about 3% w/w to about 25% w/w of the adhesive layer, said norethindrone and norethindrone acetate forming substantially no crystals in said pressure sensitive adhesive-layer when the patch is stored at a temperature of from about 25° C. to about 40° C. and a relative humidity of from about 60% to about 75% for a period of from about 3 months,
wherein upon application of the patch to the subject, the norethindrone and norethindrone acetate are transdermally delivered at a combined flux having a peak rate of at least about 2 ug/cm²/hr, that is achieved within about 60 hours after initiation of patch administration, and is followed by a subsequent flux rate of no less than about 40% of the peak flux rate until about 7 days after initiation of patch administration, thereby providing a norethindrone serum concentration sufficient to cause contraception, and is sustained for an administration period of about 7 days.

2. The transdermal matrix patch of claim 1, wherein the peak flux rate is achieved within about 24 hours after initiation of patch administration.

3. The transdermal matrix patch of claim 1, wherein the norethindrone serum concentration provided by the transdermal patch is at least about 150 pg/ml.

4. A method of providing contraception to a female subject comprising:
applying a transdermal composition as recited in any of claims 1-3 to a skin surface of the female subject.
